(19) United States
(12) Patent Application Publication
(10) Pub. No.: US 2023/0193189 A1
(43) Pub. Date: Jun. 22, 2023

(54) LIVING CELL SEPARATION SYSTEM USING ENZYMATIC DEGRADATION TREATMENT

(71) Applicant: TERUMO KABUSHIKI KAISHA, Tokyo (JP)

(72) Inventors: Eri NOGUCHI, Ashigarakami-gun (JP); Masaki MATSUMURA, Ashigarakami-gun (JP)

(73) Assignee: TERUMO KABUSHIKI KAISHA, Tokyo (JP)

(21) Appl. No.: 18/166,216

(22) Filed: Feb. 8, 2023

Related U.S. Application Data
(63) Continuation of application No. PCT/JP2021/036054, filed on Sep. 30, 2021.

(30) Foreign Application Priority Data
Sep. 30, 2020 (JP) 2020-165840

(51) Int. Cl. C12M 1/00; C12N 5/077; C12M 3/06

(52) U.S. Cl. CPC C12M 45/09; C12N 5/0658; C12M 27/16

(57) ABSTRACT

A system that easily and stably separates various living cells of interest from a living body-derived tissue, the system including: a suspension unit that prepares a suspension by adding a proteolytic enzyme solution to a living body-derived tissue based on a parameter and shaking the tissue to which the solution has been added; a measurement unit that acquires information regarding the suspension; and an analysis unit that specifies a position of living cells from the information acquired by the measurement unit. Methods for separating various living cells of interest from a living body-derived tissue are also disclosed.

### CROSS-REFERENCES TO RELATED APPLICATIONS

This application is a continuation of International Application No. PCT/JP2021/036054 filed on Sep. 30, 2021, which claims priority to Japanese Application No. 2020-165840 filed on Sep. 30, 2020, the entire content of both of which is incorporated herein by reference.

### TECHNOLOGICAL FIELD

The present disclosure generally relates to a system for separating living cells from a living body-derived tissue using an enzymatic degradation treatment.

### BACKGROUND DISCUSSION

In recent years, attempts have been made to transplant various cells for repair of damaged tissues and the like. For example, attempts have been made to use fetal cardiomyocytes, myoblast cells, mesenchymal stem cells, cardiac stem cells, ES cells, iPS cells, and the like for repair of myocardial tissue damaged by ischemic heart disease such as angina pectoris and myocardial infarction (Haraguchi et al., Stem Cells Transl Med. 2012 February; 1(2): 136-41).

As a part of such attempts, a cell structure formed using a scaffold and a sheet-shaped cell culture in which cells are formed in a sheet shape have been developed (Sawa et al., Surg Today. 2012 January; 42(2): 181-4).

For the application of the sheet-shaped cell culture to treatment, studies have been made on the use of a cultured epidermal sheet for skin damage caused by burns or the like, the use of a corneal epithelial sheet-shaped cell culture for corneal damage, the use of an oral mucosa sheet-shaped cell culture for endoscopic resection of esophageal cancer, and the like, and some of them have entered the stage of clinical application.

Myoblast cells used for such treatment are usually obtained by separating CD 56 positive cells such as myoblast cells and muscle satellite cells from skeletal muscle tissue to be transplanted. As a measure to increase a ratio of the CD 56 positive cells contained in cells separated from the skeletal muscle tissue, for example, a method is known that includes a first process involving immersing skeletal muscle tissue in a first protease solution for a prescribed time and then discarding the obtained first enzyme-treated solution and a second process involving immersing the skeletal muscle tissue resulting from the first process in a second protease solution for a prescribed time and then recovering the cells contained in the second enzyme-treated solution (JP 2007-89442 A).

### SUMMARY

In order to separate living cells from living body-derived tissue as described above, various separation methods and separation conditions have been devised. However, there are various cells constituting various living tissues, and among them, stem cells and precursor cells have a low proportion in the living tissue, and many exist in a special niche. For example, skeletal muscle tissue is composed of muscle fibers, and the parenchyma of the muscle fibers is multi-nucleus cells surrounded by a plasma membrane. However, CD 56 positive cells such as myoblast cells, which are precursor cells of the muscle fibers, are localized only between the basement membrane and the plasma membrane of the muscle fibers. Therefore, in order to separate these CD 56 positive cells from the skeletal muscle tissue of a subject, it is necessary to disrupt the skeletal muscle tissue to such an extent that the basement membrane of the muscle fibers is loosened and the CD 56 positive cells are not destroyed.

So far, separation of CD 56 positive cells from skeletal muscle tissue has been mainly performed by manual mincing and enzymatic degradation. The operation of separating myoblast cells by the enzymatic degradation treatment is complicated and takes a long time, and the determination of a cell portion collected in the enzyme treatment liquid depends on the skill of the worker, and thus the number of collected living cells among workers varies. As described above, a method of easily and stably separating various living cells from various living body-derived tissues has not yet been found.

As a result of intensive studies to solve the above-described problems, the present inventors have found that it is possible to stably separate living cells from a living body-derived tissue by acquiring information on a suspension obtained by suspending a living body-derived tissue in a proteolytic enzyme solution during enzymatic degradation treatment, and detecting a boundary between a region containing living cells of interest and other regions in the suspension. As a result of further research based on such findings, the present inventors have found that the boundary between these regions can be mechanically detected, and the entire work or a part thereof can be automated.

That is, the present disclosure relates to the following.
[1] A system that separates living cells from a living body-derived tissue, the system including:
a suspension unit that prepares a suspension by adding a proteolytic enzyme solution to the living body-derived tissue based on a parameter and shaking the tissue to which the solution has been added;
a measurement unit that acquires information regarding the suspension; and
an analysis unit that specifies a position of the living cells from the information acquired by the measurement unit.
[2] The system according to [1], further including a learning unit that extracts excess or deficiency of a parameter based on information from the analysis unit.
[3] The system according to [2], in which extracting the excess or deficiency of the parameter with the learning unit includes utilizing a sedimentation rate of the cells.
[4] The system according to [2] or [3], further including an update unit that updates the parameter based on the excess or deficiency of the parameter extracted by the learning unit.
[5] The system according to any one of [1] to [4], further including a collection unit that collects a living cell-containing solution from the suspension based on information from the analysis unit.
[6] The system according to any one of [1] to [5], further including: a second measurement unit that acquires information regarding the collected living cell-containing solution; and a second analysis unit that evaluates an amount of the living cells from the information acquired by the second measurement unit.
[7] The system according to [6], in which the learning unit further extracts excess or deficiency of the parameter based on information from the second analysis unit.
[8] The system according to any one of [1] to [7], in which the parameter includes at least one of an added amount of the proteolytic enzyme solution, a number of shaking, a shaking speed, and a standing time.
[9] The system according to any one of [1] to [8], in which the information regarding the suspension includes a turbidity of the suspension.
[10] The system according to any one of [6] to [9], in which the information regarding the collected living cell-containing solution includes a turbidity of the collected living cell-containing solution.
[11] The system of any one of [1] to [10], in which the living cells are myoblasts.

According to the system of the present disclosure, it is possible to simply, reliably, and automatically perform the operation of separating living cells from the living body-derived tissue at the time of the enzymatic degradation treatment, and it is possible not only to eliminate the variation in the number of collected living cells among workers but also to greatly reduce labor of the workers.

Another aspect of the present disclosure relates to a method for separating living cells from a living body-derived tissue. The method may involve: preparing a suspension by adding a proteolytic enzyme solution to the living body-derived tissue; shaking the suspension containing the living body-derived tissue and the proteolytic enzyme solution; acquiring information about a parameter of the suspension; identifying a position of the living cells in the suspension using the acquired information about the parameter of the suspension; and determining whether the living cells are separated from the living body-derived tissue based on the identified position of the living cells in the suspension.

### BRIEF DESCRIPTION OF THE DRAWINGS

FIG. 1 illustrates a flow diagram of an exemplary embodiment of a system disclosed herein that determines if separation of the living cells in the suspension unit is completed.

FIG. 2 illustrates a flow diagram of an exemplary embodiment of a system described herein that determines if separation of the living cells in the suspension unit is completed and determines if collection of the livings cells in the collection unit is completed.

FIG. 3 illustrates an example of components of a system that separates living cells of interest from a living body-derived tissue.

### DETAILED DESCRIPTION

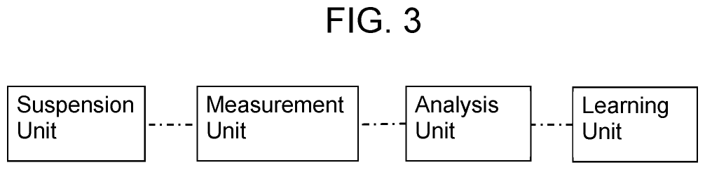

The present disclosure relates to a system that separates living cells from a living body-derived tissue, the system including, as generally shown in FIG. 3: a suspension unit that prepares a suspension by adding and/or suspending a proteolytic enzyme solution to a living body-derived tissue based on a parameter; a measurement unit that acquires information regarding the suspension; and an analysis unit that calculates a turbidity of the suspension from the information acquired by the measurement unit and specifies a position of the living cells.

In the present disclosure, the living body-derived tissue is not particularly limited as long as it is derived from a living body, and is, for example, muscle tissue, fat tissue, skin tissue, cartilage tissue, tendon tissue, ligament tissue, interstitium, vascular tissue, brain tissue, circulatory system tissue, digestive system tissue, metabolic system tissue, lymphatic system tissue, bone marrow tissue, blood, or the like, preferably muscle tissue, fat tissue, bone marrow tissue, blood, and more preferably skeletal muscle tissue. In the present invention, the living body-derived tissue may be a tissue after a mincing process.

The living cell in the present disclosure can include any living cell separated from the living body-derived tissue. Non-limiting examples thereof include cardiomyocytes, fibroblast cells, epithelial cells, endothelial cells, hepatocytes, pancreatic cells, renal cells, adrenal cells, periodontal ligament cells, gingival cells, periosteal cells, skin cells, synoviocytes, chondrocytes, and the like, and stem cells (for example, myoblasts (for example, myoblast cells) (Myoblasts include satellite myocytes), mesenchymal stem cells (for example, those derived from bone marrow, adipose tissue, peripheral blood, skin, hair roots, muscle tissue, endometrium, placenta, cord blood, and the like), tissue stem cells such as cardiac stem cells, embryonic stem cells, etc.). The living cell in the present disclosure may be a cell that is in direct contact with a plurality of membranes or localized so as to be surrounded by the membranes so as to be adjacent to the plurality of membranes between the plurality of membranes in contact with each other in a plane. In the present disclosure, examples of the living cells preferably include CD 56 positive cells in skeletal muscle tissue such as myoblast cells, or mesenchymal stem cells derived from bone marrow, adipose tissue, and peripheral blood.

In the present disclosure, cells, tissues, and the like other than the living cells that are intended to be collected from a living body-derived tissue are referred to as impurities. For example, when myoblasts and the like are separated from skeletal muscle tissue, white tissue (tendons, blood vessels, fats, etc.) and fibroblast cells become impurities.

The living body-derived tissue used in the present disclosure can be derived from any organism. Such organisms include, but are not limited to, humans, non-human primates, rodents (mice, rats, hamsters, guinea pigs, etc.), dogs, cats, pigs, horses, cows, goats, sheep, and the like. When cells separated from the living body-derived tissue are used for transplantation, the living body-derived tissue used in the present disclosure can avoid rejection by using autologous cells separated from the living body-derived tissue collected from the subject (recipient) himself/herself. However, it is also possible to use heterologous cells or homologous non-autologous cells separated using heterologous or homologous non-autologous living body-derived tissues.

In the present disclosure, the “suspension unit” is a unit where a proteolytic enzyme solution is added to a living body-derived tissue to perform an enzymatic degradation treatment. In the enzyme degradation treatment, the suspension unit suspends the living body-derived tissue and the proteolytic enzyme solution to prepare a suspension. The suspension unit includes a container that contains a living body-derived tissue, an injection unit that injects a proteolytic enzyme solution into the container, and a shaking unit that shakes the container. The injection unit is not particularly limited as long as it can control an amount of the proteolytic enzyme to be added to the living body-derived tissue. The shaking unit is not particularly limited as long as the speed, the number of times, and the like of shaking can be controlled. For example, the shaking unit may support a container containing a living body-derived tissue from below and shake the container by swinging a support surface thereof, may be a robot having an arm capable of fixing the container to a tip end and shake the container by moving the arm, or may be a unit including a commercially available shaker.

The suspension unit adds the proteolytic enzyme solution from the injection unit to the container containing the living body-derived tissue according to the parameter, and shakes the container, and then leaves a prepared suspension for a certain period of time. Examples of the parameter include an addition amount of the proteolytic enzyme solution, the number of shaking, a shaking speed, a standing time, and the like. As the container, a commercially available container for cell culture, for example, a tube, a flask, or the like can be used, but it is preferable that at least a part thereof is transparent in order to facilitate acquisition of information on the suspension in the measurement unit. The proteolytic enzyme solution may contain a proteolytic enzyme such as a collagenase or a matrix metalloprotease that degrades fibrous tissue, trypsin that separates adhesion between cells and adhesion between cells and a culture substrate, or TrypLE™ Select (Life Technologies). The proteolytic enzyme solution may contain one or more proteolytic enzymes, for example, may contain both collagenase and trypsin. The concentration of collagenase may be 0.01 to 0.25% (W/V) and the concentration of trypsin may be 0.001 to 0.25% (V/V). Specifically, a trypsin-EDTA (1×) solution (Life Technologies) can be used as trypsin, and collagenase A (Roche Applied Science), Collagenase Lyophilized (Derived from Clostridium Histolyticum, Life Technologies), and Liberase MNP-S (Roche Applied Science) can be used as collagenase.

In the present disclosure, the “measurement unit” is a unit for acquiring information on a suspension. Examples of the information on the suspension include a turbidity of the suspension, and other information suitable for identifying a position of the living cell of interest in the suspension can be obtained. As a method of measuring the turbidity of the suspension, any known method can be used, and for example, a turbidity meter using scattered light or transmitted light can be used. Such a turbidity meter using scattered light or transmitted light represents an example of a measurement unit for acquiring information on a suspension. Alternatively, instead of or in conjunction with measuring the turbidity of the suspension, images of the suspension can also be acquired using a CCD camera or the like of the suspension to locate the living cells of interest. Such a CCD camera or the like may represent another example of a measurement unit for acquiring information on a suspension.

For example, in a case where myoblasts are separated from the skeletal muscle tissue subjected to the mincing process, the impurities are fibroblast cells and the like. In such an aspect, since myoblasts and fibroblast cells have different physical properties such as size and weight, there is a difference in sedimentation rate when the suspension is left standing after being sufficiently suspended. Therefore, cells with a higher sedimentation rate gather at a bottom of the container more quickly, resulting in multiple regions with different turbidity depending on the cell type present in the suspension. In this case, how different turbidity occurs varies depending on the manner of suspension such as the shaking speed and the number of shaking, and a length of time of standing after suspension. From the above, it is possible to specify a position of myoblasts, which are living cells of interest, by a change in turbidity or an image.

In the present disclosure, the “analysis unit” is a unit that receives information from the measurement unit and specifies the position of a living cell of interest. In the present disclosure, specifying the position of a living cell of interest refers to specifying a region containing many living cells of interest in a suspension. In one aspect, the analysis unit continuously measures the turbidity of the suspension from an upper part to a lower part of the container in order to specify the position of the living cell of interest, and when the rate of change exceeds a preset threshold value, the analysis unit can detect the portion as a boundary between a region containing the living cell of interest and a region containing the impurities, and further, from the difference in physical properties between the living cell of interest and the impurities, the analysis unit can determine on which side of the boundary the living cell of interest is present, and specify the position of the living cell of interest. In another aspect, the analysis unit can acquire an image of the suspension to specify the position of the living cell of interest, detect the boundary between the region containing the living cell of interest and the region containing the impurities by analyzing the image, for example, analyzing a change in color tone of the image, or comparing the image with a previously input image, and similarly specify the position of the living cell of interest.

The analysis unit includes at least a processor (central processing unit) that receives information from the measurement unit, calculates a change rate of the acquired turbidity in order to specify the position of the living cell of interest from the information, compares the calculated value with the threshold value, and/or analyzes the image, but may further include a storage unit, a control unit, an input unit, and an output unit. The storage unit is a unit that stores the information from the measurement unit, the calculated change rate, the analyzed image, the specified position of the living cell, and the like, and includes various electronic storage media, for example, a semiconductor memory, a hard disk, and the like. The control unit is a unit that transmits a signal to the suspension unit or the like based on the specified position of the living cell or the like, and includes a signal generation circuit or the like. The input unit is a unit to which an operator of the system disclosed here, another unit of the system, or another system inputs information such as a threshold value and an image to be compared as necessary, and includes various input interfaces, for example, a unit (electric wire, optical fiber, connector, wireless communication device, and the like) that receives signals such as electricity and light from another unit, a button, a keyboard, a touch panel, and the like. The output unit is a unit that emits a predetermined signal on the basis of the calculated value, the determination of the separation state, and the like, and includes various output interfaces, for example, a unit (electric wire, optical fiber, connector, wireless communication device, and the like) that transmits a signal such as electricity or light to another unit, a monitor, a printer, an indicator lamp, a buzzer, a speech synthesizer, and the like. The input and output units may be integrated as an input/output interface, including an input interface and an output interface, and a general-purpose computer may be utilized for this purpose.

The system of the present disclosure can further include a learning unit. In the present disclosure, the “learning unit” is a unit that extracts excess or deficiency of a parameter on the basis of information from the analysis unit. The learning unit extracts excess or deficiency of the parameter by associating the parameter of the suspension unit with an output from the analysis unit regarding the change rate of the turbidity calculated by the analysis unit, the analysis of the image, the position of the living cell of interest, and the like. For example, when the change rate of the turbidity is low, the boundary between the region containing the living cell of interest and the region containing the impurities cannot be detected, and the position of the living cell of interest cannot be specified, the learning unit can determine that the number of times of shaking and the shaking speed are insufficient or the standing time is too long on the assumption that both the living cell of interest and the impurities have completely settled in the lower part of the container.

The learning unit may further include a learning storage unit. The learning storage unit is a unit that accumulates and stores a parameter of the suspension unit and information of the parameter from the analysis unit, and includes various electronic storage media similarly to the storage unit. The learning unit may extract excess or deficiency of the parameter with higher accuracy with reference to the data stored in the learning storage unit. Furthermore, the learning unit may further include a learning input unit and a learning output unit. The learning input unit is a unit where an operator of the system of the present disclosure, another unit of the system, or another system inputs information that can enable more efficient and highly accurate learning such as the enzymatic degradation treatment, the suspension environment and the like as necessary, and the learning output unit is a unit that emits a predetermined signal on the basis of the extracted excess or deficiency of the parameter. The learning input unit and the learning output unit may include interfaces similar to the input unit and the output unit, respectively.

In one aspect, the learning unit inputs information regarding a sedimentation rate of each cell contained in the suspension to the learning input unit as information that can enable highly accurate learning, thereby enabling more accurate extraction of the parameter. The information on the sedimentation rate of the cell is, for example, a diameter of the cell, a density of the cell, a density of the solvent, viscosity of the solvent, and the like, and the sedimentation rate of the cell can be calculated from these values. The learning unit calculates the sedimentation rate of each cell from the input information regarding the sedimentation rate of the cell, and adds the information to the information from the analysis unit, thereby enabling extraction of a parameter with higher accuracy.

The system of the present disclosure may further include an update unit. In the present disclosure, the “update unit” is a unit that updates a parameter on the basis of excess or deficiency of the parameter extracted by the learning unit. The update unit receives information from the learning unit, creates a new parameter reflecting the information, and updates the parameter of the suspension unit to the created parameter. The update unit may include an output interface for outputting to the suspension unit or may be integrated with the suspension unit. Alternatively, the update unit is integrated with the learning unit, and may not receive the information from the learning unit, but may create a new parameter and update the parameter of the suspension unit on the basis of the information from the analysis unit.

The system of the present disclosure may further include a collection unit. In the present disclosure, the “collection unit” is a unit that collects a region (that is, a living cell-containing solution) containing living cells of interest in the suspension based on the information from the analysis unit. The collection unit can include a new container that contains the collected living cell-containing solution of interest. Based on the output from the analysis unit such as the specified position of the living cell, the collection unit collects the living cell-containing solution of interest by, for example, sucking the solution at the position and transferring the solution to the new container. When the living cell-containing solution of interest is collected, the collection unit may similarly suck and remove the supernatant of the impurities. The output may reach the collection unit via an output unit associated with the analysis unit.

The system of the present disclosure can further include a second measurement unit and a second analysis unit. In the present disclosure, the second measurement unit is a unit that acquires information regarding the collected living cell-containing solution. Examples of the information regarding the collected living cell-containing solution include a turbidity of the collected living cell-containing solution, and other information suitable for evaluating an amount of the living cells of interest in the collected living cell-containing solution can be acquired. In the present disclosure, the second analysis unit is a unit that receives the information from the second measurement unit and evaluates an amount of the living cells of interest. In the evaluation of the amount of the living cells, a value may be calculated by converting the measured turbidity into a cell density or a cell concentration, or by comparing the turbidity or the calculated cell density or cell concentration with a previously input threshold value, for example, “suitable” or “unsuitable” may be output. In one aspect, the second analysis unit may include an output interface that outputs to the suspension unit so as to further add a proteolytic enzyme solution to the suspension obtained by collecting the living cell-containing solution, perform an enzymatic degradation treatment, and prepare a new suspension. In this case, when further collecting the living cell-containing solution from the new suspension, the collection unit can combine and mix the collected living cell-containing solution and the previously collected living cell-containing solution. In one aspect, the learning unit may be configured to further extract excess or deficiency of the parameter on the basis of the information from the second analysis unit. The measurement unit and the analysis unit may play the roles of the second measurement unit and the second analysis unit, respectively.

As described above, the components constituting the system of the present disclosure can be arranged in various manners as long as a predetermined object can be achieved, and can be combined or integrated as necessary.

Hereinafter, the system of the present disclosure will be described in more detail with reference to the drawings, which show exemplary embodiments that are in accordance with the present disclosure. The present invention is not limited to these embodiments disclosed by way of example.

In one aspect, the separation of living cells from a living body-derived tissue in the system of the present disclosure is roughly divided into two stages. That is, one stage includes suspending a living body-derived tissue in a proteolytic enzyme solution, causing a difference in turbidity in the suspension, separating a region containing living cells of interest (that is, a living cell-containing solution) and impurities (unintended cells or tissues, living body-derived tissues unreacted with a degrading enzyme, and the like), and specifying a position of the living cells of interest, and the other stage includes collecting the separated living cell-containing solution, and evaluating an amount of living cells of interest in the solution from the turbidity of the solution. Here, in the former stage, when the living body-derived tissue is skeletal muscle tissue and the living cell of interest is myoblast, the myoblast and the fibroblast cell contained in the impurities are classified by comparing physical properties such as size and weight. Furthermore, when it is determined in the evaluation in the latter stage that the collection of living cells of interest has not been completed, a proteolytic enzyme solution can be further added to and suspended in the residue of the suspension in which the living cell-containing solution has been collected, and thereafter, the living cell-containing solution can be collected, and it can be mixed with a solution containing living cells previously collected, and the amount of living cells of interest in the solution can be evaluated again from the turbidity of the solution after the mixing. These operations may be repeated until it is determined that the collection of the living cells of interest has been completed in the evaluation of the amount of the living cells in the collected living cell-containing solution.

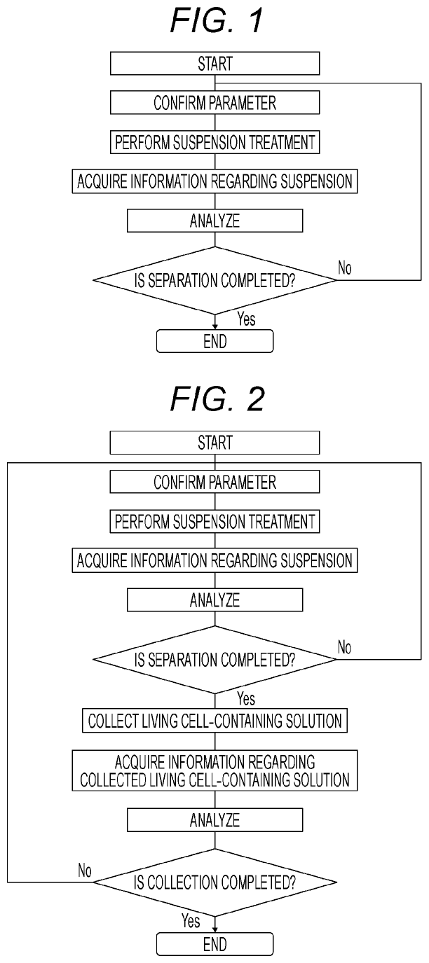

FIG. 1 illustrates a flow diagram of a stage of separating a live cell-containing solution and impurities in one aspect of the system of the present disclosure. As shown in FIG. 3, the system can include a suspension unit, a measurement unit and an analysis unit, each of which is described in more detail below. The system may also include a learning unit. In this embodiment disclosed as an example, when the system receives a start instruction, the suspension unit starts the enzymatic degradation treatment according to the input parameter. In the enzymatic degradation treatment, a proteolytic enzyme is added to and suspended in a living body-derived tissue to prepare a suspension. The measurement unit acquires information regarding the suspension, for example, the turbidity of the suspension. The analysis unit specifies a position of the living cell of interest from the information regarding the suspension. Within a proper suspension, a boundary arises between a region containing the living cells of interest and a region of impurities, and the boundary can be confirmed and the position of the living cells of interest can be specified, for example, by a rate of change in turbidity. In one aspect of the present disclosure, the analysis unit can determine that the separation in this aspect is completed when the rate of change in turbidity of the suspension exceeds a preset threshold value. When the separation is completed, the system of the present disclosure can output a separation complete signal to end the flow. If the suspension is not suitable and the analysis unit determines that the separation has not been completed, the analysis unit can output a signal to the suspension unit and the suspension can be performed again by the suspension unit, and in this case, the parameter of the suspension can be changed based on the information from the analysis unit. The parameter may be changed by the operator when inputting it into the suspension unit. In one aspect, the system of the present disclosure may further include the learning unit and the update unit, a signal may be outputted from the analysis unit to the learning unit, and in these units, a parameter may be changed and applied (updated) to the suspension unit.

FIG. 2 illustrates a flow diagram in one aspect of a system of the present disclosure, including collecting the localized living cells as a living cell-containing solution and evaluating an amount of living cells of interest in the solution from the turbidity of the solution. Also in the present aspect, similarly to the flow in FIG. 1, when the system receives a start instruction, the suspension unit starts the enzymatic degradation treatment according to the input parameter to prepare a suspension, the measurement unit acquires information regarding the suspension, for example, the turbidity of the suspension, and the analysis unit specifies a position of the living cell of interest from the information regarding the suspension. In one aspect of the present disclosure, the analysis unit can determine that the separation in this aspect is completed when a change rate of the turbidity of the suspension exceeds a preset threshold value. When the analysis unit determines that the separation is completed, the analysis unit can output a signal to the collection unit and the collection unit can collect the living cell-containing solution. With respect to the collected living cell-containing solution, information regarding the collected living cell-containing solution is acquired by the second measurement unit, and the second analysis unit evaluates an amount of the living cells from the information acquired by the second measurement unit. The second analysis unit can determine that the collection in this aspect is completed when the amount of the living cells is more than or equal to a preset threshold value. When the collection is completed, the second analysis unit outputs a collection completion signal and can terminate the flow. When the amount of the living cells falls below a preset threshold value, the second analysis unit outputs a signal to the suspension unit, and a proteolytic enzyme solution is further added to the remaining residue of the suspension from which the living cell-containing solution has been collected, and an enzymatic degradation treatment is performed to prepare a new suspension. For the new suspension, the measurement unit acquires information regarding the suspension, the analysis unit specifies the position of the living cell from the information acquired by the measurement unit, and the collection unit collects the living cell-containing solution from the new suspension based on the information from the analysis unit, and mixes it with the previously collected living cell-containing solution to obtain a new living cell-containing solution. The second measurement unit and the second analysis unit acquire information on the new living cell-containing solution and evaluate the amount of the living cells. In this way, the separation and collection of the living cells of interest can be repeated until the amount of the living cells exceeds the threshold value and the separation is complete. Furthermore, in the present aspect, the parameter of the suspension can be changed on the basis of the information from the analysis unit and the second analysis unit, and the change may be inputted by an operator of the system or may be made by the learning unit and the update unit.

Although one aspect of the system of the present disclosure has been described above, it should be understood that various aspects other than the above are possible. Therefore, various aspects obtained by modifying the above aspects without departing from the spirit of the present invention are also included in the scope of the present invention, and such modifications are understandable to those skilled in the art.

What is claimed is:

1. A method for separating living cells from a living body-derived tissue, the method comprising:
preparing a suspension by adding a proteolytic enzyme solution to the living body-derived tissue;
shaking the suspension containing the living body-derived tissue and the proteolytic enzyme solution;
acquiring information about a parameter of the suspension;
identifying a position of the living cells in the suspension using the acquired information about the parameter of the suspension; and
determining whether the living cells are separated from the living body-derived tissue based on the identified position of the living cells in the suspension.

2. The method of claim 1, further comprising:
collecting the living cells that have been separated from the living body-derived tissue and forming a living cell-containing solution using the collected living cells;
acquiring information on a parameter of the living cell-containing solution;
identifying a position of the living cells in the living cell-containing solution; and
determining that separation of the living cells is complete when an amount of living cells in the living cell-containing solution is more than or equal to a preset threshold value.

3. The method according to claim 1, wherein the living body-derived tissue is derived from muscle tissue, fat tissue, skin tissue, cartilage tissue, tendon tissue, ligament tissue, interstitium, vascular tissue, brain tissue, circulatory system tissue, digestive system tissue, metabolic system tissue, lymphatic system tissue, bone marrow tissue or blood.

4. The method according to claim 1, wherein the living cells of interest are cardiomyocytes, fibroblast cells, epithelial cells, endothelial cells, hepatocytes, pancreatic cells, renal cells, adrenal cells, periodontal ligament cells, gingival cells, periosteal cells, skin cells, synoviocytes, chondrocytes or stem cells.

5. The method according to claim 1, wherein the proteolytic enzyme solution contains a collagenase, a matrix metalloprotease, trypsin or any combination thereof.

6. The method according to claim 1, wherein the living cells of interest are myoblasts.

7. A system that separates living cells of interest from a living body-derived tissue, the system comprising:
a suspension unit that prepares a suspension by adding a proteolytic enzyme solution to the living body-derived tissue and that shakes the suspension to which the tissue has been added;
a measurement unit that acquires information on a parameter of the suspension; and
an analysis unit that specifies a position of the living cells of interest from the information on the parameter acquired by the measurement unit.

8. The system according to claim 7, further comprising a collection unit that collects a living cell-containing solution from the suspension based on information from the analysis unit.

9. The system according to claim 8, further comprising: a second measurement unit that acquires information regarding the collected living cell-containing solution; and a second analysis unit that evaluates an amount of the living cells of interest from the information acquired by the second measurement unit.

10. The system according to claim 7, wherein the parameter includes at least one of an added amount of the proteolytic enzyme solution, a number of shaking, a shaking speed, and/or a standing time.

11. The system according to claim 7, wherein the information regarding the suspension includes a turbidity of the suspension.

12. The system according to claim 11, wherein the information regarding the collected living cell-containing solution includes a turbidity of the collected living cell-containing solution.

13. The system according to claim 7, wherein the living cells of interest are myoblasts.
